(19) United States
(12) Patent Application Publication
(10) Pub. No.: US 2017/0000635 A1
(43) Pub. Date: Jan. 5, 2017

(54) Wearable Communication Board

(71) Applicant: Halley P. Profita, Boulder, CO (US)

(72) Inventor: Halley P. Profita, Boulder, CO (US)

(21) Appl. No.: 14/522,582

(22) Filed: Oct. 23, 2014

Related U.S. Application Data
(60) Provisional application No. 61/894,417, filed on Oct. 23, 2013.

(51) Int. Cl. A61F 4/00; G06F 1/16

(52) U.S. Cl. CPC A61F 4/00; G06F 1/163

(57) ABSTRACT

A wearable communication board can be wrapped around a user's thigh, arm, etc. It presents the user a number of button indicators on an interactive panel. When pressed, the button indicators activate an underlying button which causes a pre-recorded word/sound to be played. This grants the user the ability to produce verbal output so that they can communicate with others. The board is lightweight, flexible, and easily transportable as opposed to most communication boards. It is customizable as button indicators and corresponding sounds can be easily swapped and/or updated.

### CROSS-REFERENCE TO RELATED APPLICATIONS

This application claims the benefit of U.S. Provisional Patent No. 61/894,417 entitled WEARABLE COMMUNICATION BOARD and filed on Oct. 23, 2013, which is specifically incorporated by reference herein for all that it discloses and teaches.

### TECHNICAL FIELD

The present invention relates generally to the field of assistive devices for the communicationally challenged; and more particularly, to an electronic textile wearable communication board.

### BACKGROUND

The field of art has known a number of different communication-assistance devices for those individuals with autism or other verbal disabilities. For example, large lap-boards are available that have a plethora of buttons that create verbal output. However, such boards are large and quite cumbersome, and are therefore neither easily portable, nor able to be carried by communicationally challenged individuals when riding horses, undergoing other therapy, or most any physical activity.

What is needed is a lightweight, comfortable, and wearable communication board, so that the board moves with the user and only requires one hand for verbal output.

### SUMMARY

One embodiment of the present invention comprises a wearable communication board that fits snugly on the user and grants them verbal output so that they can communicate with therapists, family members, friends, and others around them. Most communication boards are large, cumbersome, and heavy and require a user to hold onto the board with one hand while pointing at the desired symbol with the other. The present invention replaces the cumbersome interaction necessitated by standard communication boards by making them lightweight, comfortable, and wearable, so that they move with the user and only require one hand for verbal output.

The invention can be made mostly of fabric or other similarly flexible material. The wearable communication board is generally flat and preferably elongated so that it can be attached and securely worn on the user's thigh, arm, torso, waist, etc. The communication board itself has a plurality of smaller images that serve as buttons. When triggered, the buttons cause the board to produce verbal output of what the user is trying to communicate.

The communication board can have a relatively flat speaker on the top. The device can be customized for children, thus having a more playful, home construction feel to it than a high-tech, adult-oriented device. Regardless of the customized look, the device has a plurality of buttons attached to an mp3 or similar microcontroller with pre-recorded sound tracks. Each button will trigger a respective recording depending on what the user is trying to say. The recording is heard through a thin speaker located on the anterior surface of the device. The buttons use symbols to indicate what recording that button will trigger.

The buttons can be constructed using conductive thread that is embroidered or sewn into a non conductive fabric platform. Foam or batting is then placed in between the two pieces of fabric embroidered/sewn with conductive thread (forming a striated button). The two conductive sides of the fabric then face each other, and are separated by the batting. When a user presses the button, the two conductive contacts touch, completing the circuit and signaling the device to produce the desired sound. In this manner, a wearable communication board assists a user in verbally communicating what they wish to say simply through the pressing of a button on a flexible, soft, wearable communication board. Also, the sounds/words can be pre-recorded by the user himself/herself so their own voice is played back when the board is activated.

### BRIEF DESCRIPTION OF THE DRAWINGS

The aforementioned and other features and objects of the present invention and the manner of attaining them will become more apparent and the invention itself will be best understood by reference to the following descriptions of a preferred embodiment and other embodiments taken in conjunction with the accompanying drawings, wherein:

FIG. 1 illustrates a top plan view of an exemplary embodiment of a wearable communication board;

FIG. 2 illustrates a bottom plan view of an exemplary embodiment of a wearable communication board;

FIG. 3 illustrates a bottom plan view of the right portion of an exemplary embodiment of a wearable communication board with the back cover removed;

FIG. 4 illustrates a bottom plan view of the left portion of an exemplary embodiment of a wearable communication board with the back cover removed;

FIG. 5 illustrates a side elevation exploded view of an exemplary embodiment of a wearable communication board showing one embodiment of the layers that can be used to construct the button portions;

FIG. 6 illustrates a top plan view of an exemplary embodiment of a plurality of indicia that indicate the functionality of the underlying buttons that can be incorporated into a wearable communication board; and

FIG. 7 illustrates a top plan view of an exemplary embodiment of a wearable communication board.

### DETAILED DESCRIPTION

The wearable communication board permits a user with a speech impairment to give verbal cues or commands when participating in a physical activity. The device is a small-scale, fabric-based communication device with audio output designed to be worn during physical activity. The wearable communication board is an easy to produce, low-cost solution that gives a user the ability of speech on the go.

For an individual with Autism, a wearable system is ideal as many times the user will leave something behind or run away from objects. Wearability ensures that the system will remain securely with the user at all times. Many electronic communication boards consist of bulky, heavy, or hard-wired electronics. Such systems are clumsy and extremely difficult or impossible to utilize during physical activities. The wearable communication board is predominantly constructed of electronic textiles, making the system both functional and soft for wearability. The system is also lightweight so as not to shift or cause any discomfort to the user.

Current communication systems that are used during physical therapy, if any, are low-tech and paper-based (commonly a hip pack or wrist cards). While these systems offer some options for a user to communicate, hip packs are hard to reach symbol options (while possibly interfering with the physical activity). Furthermore, usage of a low-tech device requires the caregiver to constantly pay attention for visual confirmation. An electronic verbal system allows a user to press dedicated buttons that correspond to pre-recorded phrases, allowing the caregiver to hear from a distance what the user is trying to communicate.

The system is stable enough to withstand the constant jostling physical activities such as horse riding. The materials used are able to withstand the elements, such as dirt and sweat and can be at least somewhat water resistant. Neoprene or other similar material can be used to form the outer, protective layer(s) of the device.

The system is easy to don and doff, stays on well, and is easy for a user or caregiver to operate. Large physical buttons that are easy for the user to locate both visually or non-visually are ideal. The system is also operable with one hand instead of two, permitting for the other hand to remain available for physical activities (such as holding horse reigns) at all times.

This system is not visually or technically overwhelming, and can also be personally programmable so that a user could record his or her own words or phrases for customization.

The thigh was chosen as the ideal location for the system as the device can easily be strapped to this area by the user without obstructing many physical activities (such as horseback riding). The thigh location permits the system to remain out of the immediate way, while still staying within arm's reach of the user.

Many electronic communication devices are costly, making their purchase a much more substantial investment (with even more incurred costs should the system fail). A design goal of this system was to construct it at a lower cost, with replaceable parts that would not be too much of a financial burden should a part of the system break and need to be repaired or replaced. While this system was designed to be used specifically for horse therapy, it could be customized for other physical therapy embodiments. An arm system for adaptive skiing could be another ideal usage, for example.

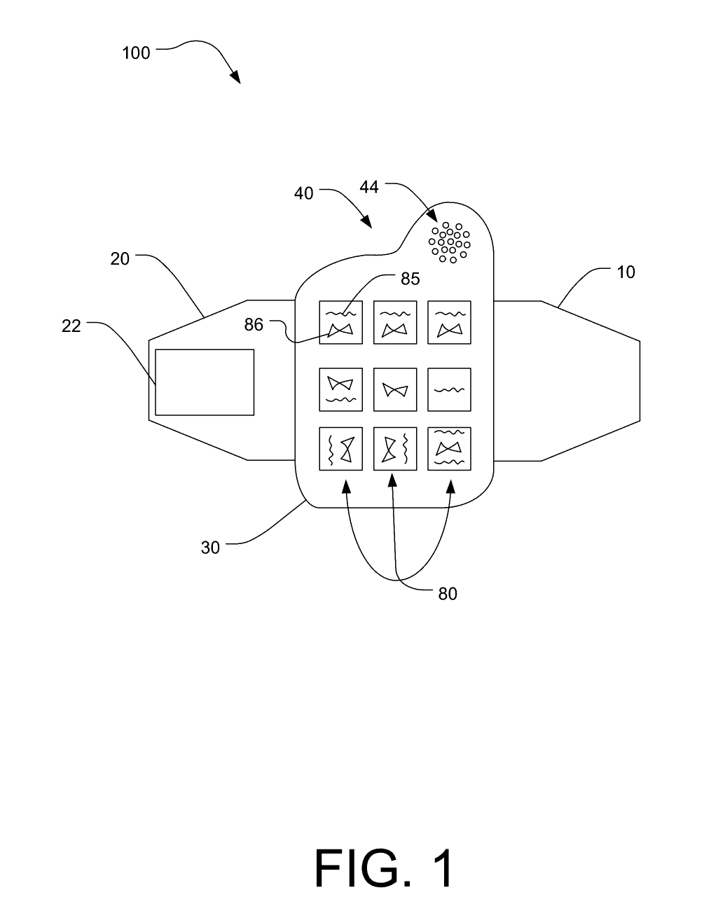

Referring now to the drawings, FIG. 1 illustrates a top perspective view of an exemplary embodiment of a wearable communication board 100. The board 100 has a plurality of button indicators 80 that can be pressed by the user in order to activate the underlying buttons. Button activation causes the system to generate pre-assigned sounds. The interactive panel 30 of the wearable communication board 100 forms the main body and can contain some of the plurality of button indicators 80 on its exterior display surface which is visible in FIG. 1 as item 30.

The button indicators 80 can have a plurality of indicia 85 and/or a plurality of graphic symbols 86 that the user can interpret in order to indicate to the user the sound that will be played when the underlying button is activated. For example, in FIG. 1, the top left button indicator can have indicia “Go” and/or a graphical symbol of a green traffic light displayed thereon, and when pressed, the wearable communication board could verbally say “Go”. The other button indicators can also be programmed to correspond with sounds related to their symbols/indicia such that when the underlying buttons are activated, the board 100 can generate corresponding sounds/words, such as: “More”, “Fun”, “Giddy Up”, “Thirsty”, “Stop”, “Bathroom”, “Tired”, “Pain”, etc.

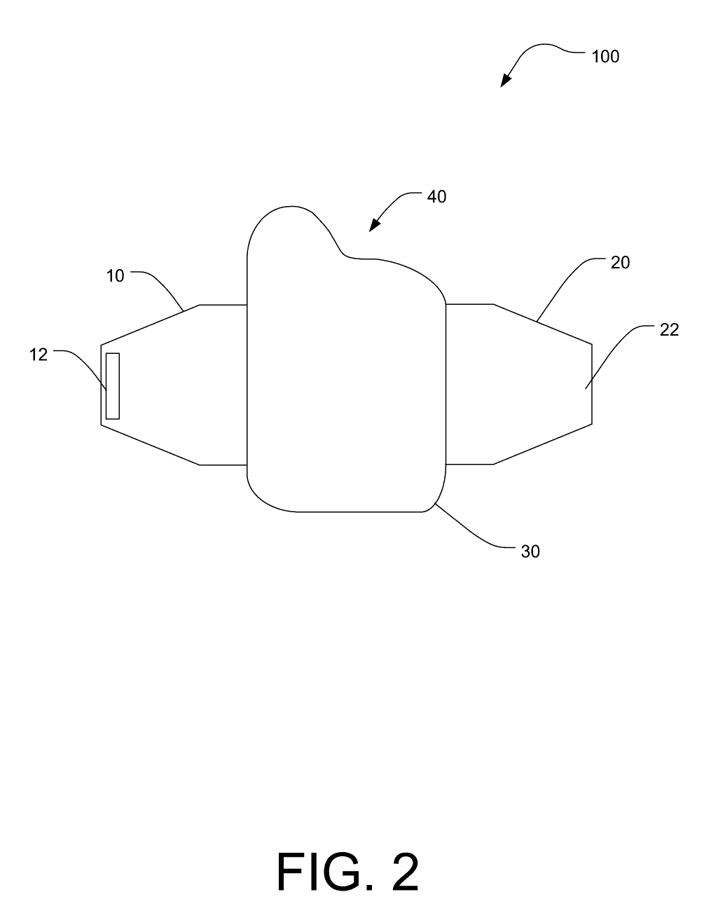

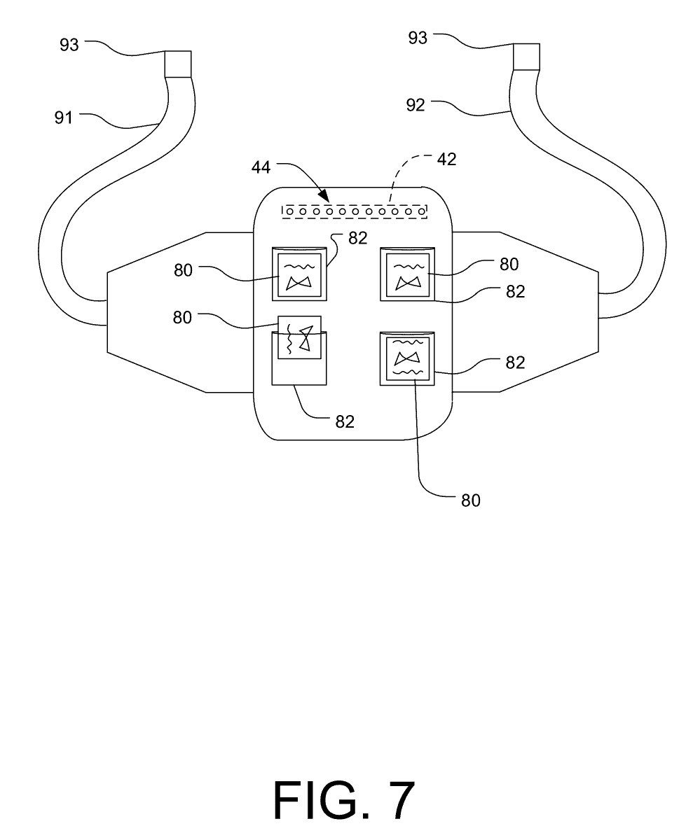

The left band 20 and right band 10 that encircle the user's body part (in the embodiment of FIG. 1, the thigh) are shown. The bands 10 and 20 simply wrap around and then attach to one another via a plurality of attachment means 22 and 12 which can be hook and loop material or other similar easy on/off attachment means (see FIG. 2, item 12 for corresponding attachment means 12 that can be connected to attachment means 22 shown in FIG. 1 when the board 100 is wrapped around a user's thigh, arm, etc.). In other embodiments, larger sizes of bands 10 and 20 can be used, as can a plurality of extension connectors (see FIG. 7, items 91 and 92).

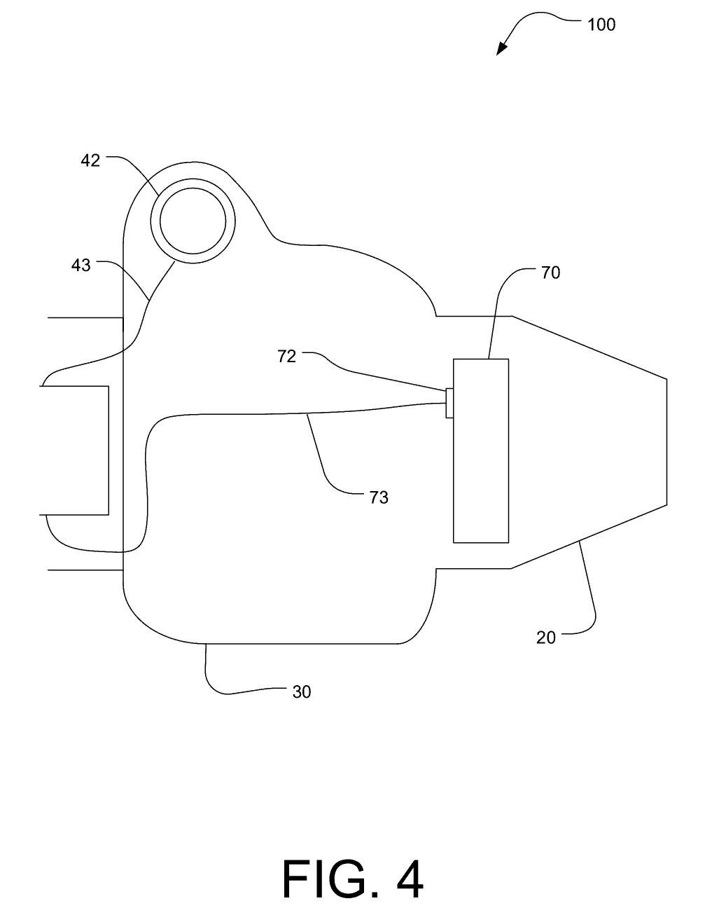

The relatively flat speaker 42 is not shown in FIG. 1, but the speaker ports 44 are visible. See FIG. 4, item 42 for a depiction of a generally round, flat speaker 42. In other embodiments, the speaker ports 44 may not be included or they can be configured differently. The speaker 42 can be located in the speaker flap 40 as shown in FIG. 1. In other embodiments, the speaker 42 can be configured differently and/or located elsewhere. For example, a long, generally rectangular speaker 42 is shown in FIG. 7.

The system is designed to have an internal power source with a power cord connected to the microcontroller. In one embodiment, the power source can be an impact-resistant, possibly rechargeable power source such as a battery pack. In another embodiment, an external power source can be alternatively connected and used as needed.

FIG. 2 illustrates a bottom perspective view of an exemplary embodiment of a wearable communication board 100. In this view, the neoprene or similar material backing is shown. When the wearable communication board 100 is attached to the user, it becomes form fitting and wraps around the thigh (or other body part). The left and right bands 20 and 10 are shown from the bottom view as are the bottom or back of the interactive panel 30. The plurality of attachment means 12 is shown in FIG. 2, see also FIG. 1, item 22 for a corresponding attachment means.

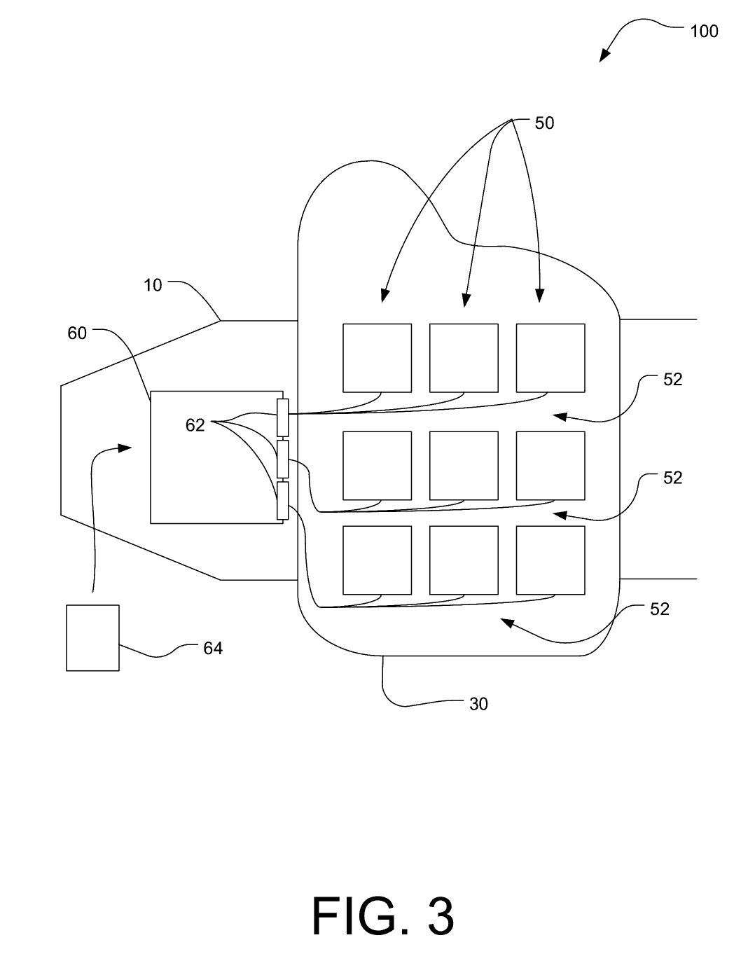

FIG. 3 illustrates a bottom perspective view of the right portion of an exemplary embodiment of a wearable communication board 100 with the back cover removed. Here, the plurality of buttons 50 can be seen from behind and the plurality of button connections 52 to the microcontroller circuit board 60 are shown. The plurality of button connections 52 connect to the microcontroller 60 via a plurality of input ports 62 on the microcontroller 60.

Note that a removable memory device 64 can be utilized with the microcontroller 60. In one embodiment, the removable memory device 64 can contain sound tracks that correspond to each of the buttons 50. Thus, when a user presses button 1, then sound 1 would be played by the microcontroller 60; a press of button 2 would cause sound 2 to play, etc. In FIG. 3, a large arrow is shown pointing from the memory device 64 to the microcontroller 60 to indicate that the memory device 64 can be inserted into the microcontroller 60. In alternate embodiments, the memory device 64 may not be removable and instead can be reprogrammed while in place within the microcontroller 60. Thus, in some embodiments, the board can have new sounds added via a replaceable memory device 64, or the memory device 64 can be on-board and can be changed or reprogrammed as needed when the board 100 needs to play new sounds. The plurality of button indicators 80 can similarly be changed when new sounds are needed or when otherwise desired. For one way in which the plurality of button indicators 80 can be swapped, see FIG. 7.

FIG. 4 illustrates a bottom perspective view of the left portion of an exemplary embodiment of a wearable communication board 100 with the back cover removed. Here, the speaker 42 is visible as is the speaker connector 43, which connects the speaker 42 to the microcontroller 60. In some embodiments, wireless technology can be used in place of a speaker connector 43.

Also visible in FIG. 4 is an internal power source 70 located in the left band 20 that is connected to the microcontroller 60 via a power cord 73 attached to the power port 72 on the power source 70. In other embodiments, the internal power source 70 can be placed in alternate locations without departing from the scope of the invention.

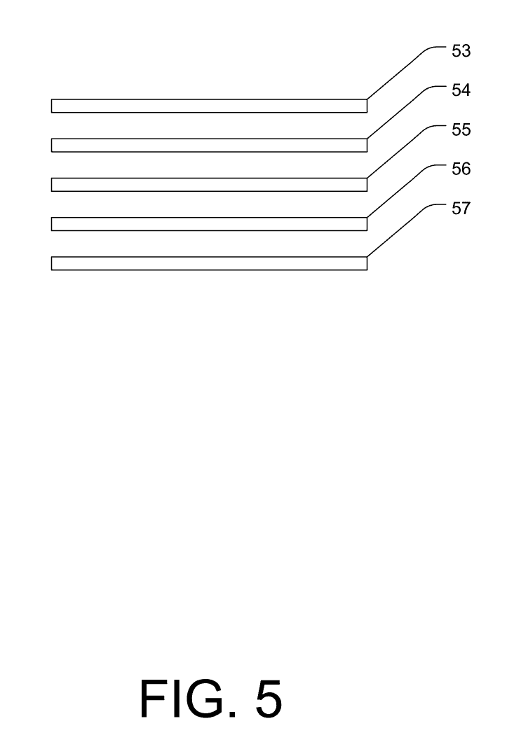

FIG. 5 illustrates a side elevation exploded view of an exemplary embodiment of a wearable communication board 100 showing one embodiment of the layers that can be used to construct the plurality of buttons 50. In the embodiment shown in FIG. 5, a first cushion layer 53 is utilized followed thereunder by a first conductive fabric layer 54. A second conductive fabric layer 56 is placed under the first conductive fabric layer 54 and is separated therefrom by a layer of perforated material 55. The perforated materials 55 can be perforated foam or other similar materials. The final layer can be a second cushion layer 57; the first and second cushion layers 53 and 57 cushion the button and also insulate the first and second conductive fabric layers 54 and 56. When the button is pressed, the conductive fabric layers 54 and 56 are allowed to touch through the perforations in the perforated material 55 and thus close the circuit. The button push is then sensed by the microcontroller and the corresponding sound is played. In other embodiments, other means of constructing a button are contemplated.

In the embodiment of FIG. 5, protective cushion layers 53 and 57 can be made from neoprene with the next layers inwards being composed of conductive fabrics. Between the first and second conductive fabric layers 54 and 56 can be batting or perforated foam material that blocks a connection between the conductive fabric layers 54 and 56 until they are pressed firmly together. The resulting connection then generates a signal which is sent to the microcontroller circuit board, which then causes a sound to play on the attached speaker.

In another embodiment, conductive thread can be embroidered or sewn into a non conductive fabric platform. Foam or batting could be placed in between the two pieces of fabric embroidered/sewn with conductive thread (forming a striated button); the two conductive sides of the fabric would be facing each other, separated by the batting. When a user presses the button together, the two conductive contacts touch, completing the circuit and signaling the generation of a sound.

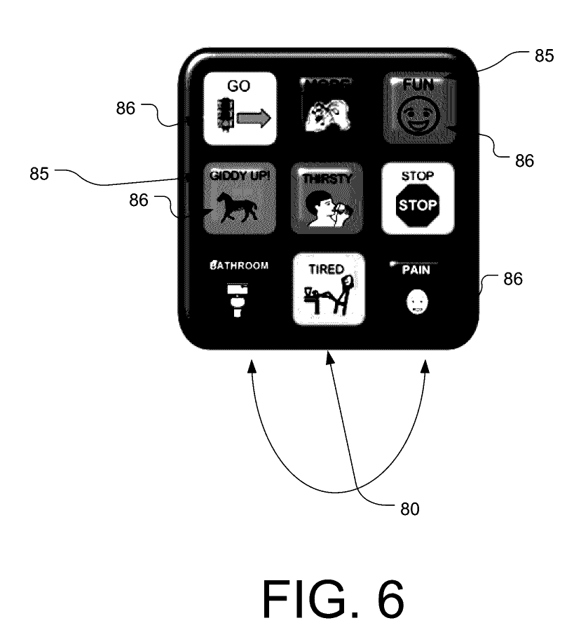

FIG. 6 illustrates a top plan view of an exemplary embodiment of a plurality of button indicators 80 that can be pressed by the user in order to activate the underlying buttons. The button indicators 80 can have a plurality of indicia 85 and/or a plurality of graphic symbols 86 that the user can interpret in order to indicate to the user the sound that will be played when the underlying button is activated. The embodiment shown in FIG. 6 is simply a formalized view of the previously described button indicators 80. The number of button indicators 80 shown is nine, in other embodiments more or fewer button indicators (and underlying buttons) are contemplated.

FIG. 7 illustrates a top plan view of an exemplary embodiment of a wearable communication board 100. Here a plurality of extension connectors 91 and 92 are shown attached to the left and right bands 20 and 10. These allow the board 100 to be worn by people with larger thighs, arms, etc. Also, the device can be placed around a horse's neck, the back of a car seat, or any other location that is needed. Visible at the ends of the extension connectors 91 and 92 are attachments 93. The attachments 93 can be buckles, hook and loop, snaps, magnets, or any other suitable attachments for connecting the extension connectors 91 and 92 together. Note that in this embodiment, only four button indicators 80 are shown.

Also shown in FIG. 7 is a plurality of button indicator pockets 82. The pockets 82 allow the button indicators 80 to be interchanged in the board 100 as desired. In other embodiments, other ways of changing button indicators 80 are contemplated.

While particular embodiments of the invention have been described and disclosed in the present application, it should be understood that any number of permutations, modifications, or embodiments may be made without departing from the spirit and scope of this invention. Accordingly, it is not the intention of this application to limit this invention in any way except as by the appended claims.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless the above “Detailed Description” section explicitly defines such terms. Accordingly, the actual scope of the invention encompasses not only the disclosed embodiments, but also all equivalent ways of practicing or implementing the invention.

The above detailed description of the embodiments of the invention is not intended to be exhaustive or to limit the invention to the precise embodiment or form disclosed herein or to the particular field of usage mentioned in this disclosure. While specific embodiments of, and examples for, the invention are described above for illustrative purposes, various equivalent modifications are possible within the scope of the invention, as those skilled in the relevant art will recognize. Also, the teachings of the invention provided herein can be applied to other systems, not necessarily the system described above. The elements and acts of the various embodiments described above can be combined to provide further embodiments.

In light of the above “Detailed Description,” the Inventor may make changes to the invention. While the detailed description outlines possible embodiments of the invention and discloses the best mode contemplated, no matter how detailed the above appears in text, the invention may be practiced in a myriad of ways. Thus, implementation details may vary considerably while still being encompassed by the spirit of the invention as disclosed by the inventor. As discussed herein, specific terminology used when describing certain features or aspects of the invention should not be taken to imply that the terminology is being redefined herein to be restricted to any specific characteristics, features, or aspects of the invention with which that terminology is associated.

While certain aspects of the invention are presented below in certain claim forms, the inventor contemplates the various aspects of the invention in any number of claim forms. Accordingly, the inventor reserves the right to add additional claims after filing the application to pursue such additional claim forms for other aspects of the invention.

The above specification, examples and data provide a description of the structure and use of exemplary implementations of the described articles of manufacture and methods. It is important to note that many implementations can be made without departing from the spirit and scope of the invention.

What is claimed is:

1. A wearable communication board, comprising:
a plurality of buttons mounted within an interactive panel;
the interactive panel having a display surface whereon a plurality of button indicators are mounted;
the plurality of button indicators each corresponding to an underlying one of the plurality of buttons;
the plurality of button indicators each having at least one of a plurality of indicia and a plurality of graphic symbols thereon that are displayed to a user and indicate what will be played when the underlying corresponding button is pressed;
a left band connected to a left side of the interactive panel and a right band connected to a right side of the interactive panel;
a microcontroller within one of the interactive panel, the left band and the right band;
a sound generating device within one of the interactive panel, the left band and the right band;
a power source within one of the interactive panel, the left band and the right band;
the left band and the right band having thereon a plurality of attachment means adapted such that the wearable communication board can be wrapped around a body part of the user and secured thereon;
the microcontroller connected to the power source, to the sound generating device, and to the plurality of buttons and adapted such that when each of the buttons is pressed, a corresponding sound is sent to the sound generating device where it is played audibly to the user

2. The wearable communication device of claim 1 further comprising a plurality of button indicator pockets adapted to allow the interchanging of the plurality of button indicators.
